(19) United States
(12) Patent Application Publication
(10) Pub. No.: US 2006/0003401 A1
(43) Pub. Date: Jan. 5, 2006

(54) Method for preparing a water-soluble carbon nanotube wrapped with self-assembly materials

(76) Inventors: Sang Yup Lee, Daejeon (KR); Hee Tae Jung, Daejeon (KR); Seok Jae Lee, Daejeon (KR); Jong Pil Park, Gyeonggido (KR); Tae Jung Park, Daejeon (KR); Jong Hyun Choi, Daejeon (KR)

Correspondence Address: INTELLECTUAL PROPERTY / TECHNOLOGY LAW, PO BOX 14329, RESEARCH TRIANGLE PARK, NC, 27709, US

(21) Appl. No.: 10/918,909

(22) Filed: Aug. 16, 2004

(30) Foreign Application Priority Data
Nov. 27, 2003 (KR) 10-2003-0084888

(51) Int. Cl. C12Q 1/04; C12M 1/34

(52) U.S. Cl. 435/34; 435/287.1

(57) ABSTRACT

A water-soluble carbon nanotube (CNT) wrapped with self-assembly material, and a method for preparation thereof, in which a mixture of the self-assembly material and CNT is provided, and the mixture induces a self-assembly of the self-assembly material on the CNT, thereby wrapping the CNT with the self-assembly material. Water-soluble CNT wrapped with self-assembly material is readily prepared by this method. The CNT wrapped with self-assembly material has excellent applicability in comparison with usual CNT because of its water solubility. Biosensors can be fabricated by selectively attaching to the CNT wrapped with self-assembly material, receptors that bind to or react with target biomaterials or organic compounds.

### BACKGROUND OF THE INVENTION

1. Field of the Invention

The present invention relates to a water-soluble carbon nanotube (CNT) wrapped with a self-assembly material, a method of making same, and a biosensor in which a receptor binding to or reacting with a bio-substance is attached selectively, to the water-soluble CNT wrapped with self-assembly material.

2. Background of the Related Art

Carbon nanotube is an allotrope of carbon, which abundantly exists on the earth. Carbon nanotube is a tubular material in which a carbon atom is connected with other carbon atoms to form a hexagonal honeycomb structure. Its diameter is in the nanometer range (1/10⁹ meter). CNT is known to have excellent mechanical properties, electrical selectivity, field emission properties and highly efficient hydrogen storage properties and be new and almost defect-free of all the existing materials.

Therefore, such CNT shows unlimited applicability in the fields of electron emitters, vacuum fluorescent displays (VFD), white luminous sources, field emission displays (FED), lithium ion secondary battery electrodes, hydrogen storage fuel cells, nano-wires, nano-capsules, nano-tweezers, AFM/STM tips, single electron device, gas sensors, medical engineering microscopic parts, high functional complex, etc.

Because of their properties including excellent structural rigidity and chemical stability, the ability to act as both a conductor or semiconductor and the property of a large aspect ratio (ratio of length to diameter), CNT exhibits great applicability in flat panel displays, transistors, and energy reservoirs, and various nanosize electron devices (Dai, H., Acc. Chem. Res., 35:1035, 2002).

Meanwhile, many applications with CNT in the bioengineering field have recently appeared. There is shown the possibility of application of CNT to biosensors, such as glucose biosensors, the detection of protein, the detection of a certain DNA sequence and the like (Sotiropoulou, S. et al., Anal. Bioanal. Chem., 375:103, 2003; Chen, R. J. et al., PNAS, 100:4984, 2003; Dai, H. et al., Anal. Bioanal. Chem., 375:287, 2003). Detecting bio-substances in a CNT-based multiplayer can allow an increase in the amount being immobilized of bio-substances such as DNA, and increase detection sensitivity to the bio-substances, due to the large surface area and high electrical conductivity of CNT.

However, currently known CNT materials are insoluble in all organic solvents and thus show limitations in many applications (Bochrath, M., Science, 275:1922, 1997). Moreover, this insoluble property hinders the understanding of the chemical property of CNT at the molecular level (Chen, J. et al., Science, 282:95, 1998).

Recently, there have been performed studies to increase the solubility of CNT by covalent bond, non-covalent bond and micelle techniques using polymers, natural polymers, carbohydrates, and peptides (O'Connell, M. J. et al., Chem. Phy. Lett., 342:265, 2001; Chen, J., et al., JACS, 124:9034, 2002; Mitchell, C. A. et al., Macromolecules, 35:8825, 2002; Kang, Y. et al., JACS, 125, 5650, 2003; Wang et al., Analyst., 127:1353, 2002; Star and Stoddart, Macromolecules, 35:7516, 2002; Bandyopadhyaya et al., Nano Lett., 2: 25, 2002; Pantarotto et al., Chemistry & Biology, 10:961, 2003).

There was shown the possibility of applying CNT as biosensors by binding or wrapping CNT with carbohydrates by physical adsorption (Wang, Z. et al., Analyst, 127:1353, 2002; Star, A. et al., Angew. Chem. Int. Ed., 41:2508, 2002). However, such methods have fatal shortcomings in that not only their binding force to CNT is weak, also it is difficult to precisely control the orientation of carbohydrates bound to CNT, since carbohydrates are simply bound to the surface of CNT by physical adsorption or van der Waals attraction (Chambers, G. et al., Nano Lett., 3:843, 2003).

Recently, SLP (surface layer protein) having a property of excellent self-assembly has been an object of intensive interest in the field of nanobiotechnology. The most important characteristic of SLP is a crystalline array structure. SLP is found in various gram negative and gram positive bacteria or Archae and the like (Pum and Sleyter, Nanotechnol., 17:8, 1999). SLP comprises 1, 2, 4, 6 subunits to construct a symmetrical lattice of a sloping structure, a rectangular structure or a hexagonal structure. Lattices are located at regular intervals of 2.5˜35 nm. Each subunit has the ability of self-assembling by non-covalent protein-protein bonding under the various conditions of solution (Sleytr et al., Trend. Microbiol., 7:253, 1999; Gyorvary et al., Nano Lett., 3:315, 2003). However, it has been reported that such crystalline array structure can be rarely found in usual conditions and also such structure is very weak as the non-covalent protein-protein bond. In self-assembling with such orientation, SLPs derived from some bacteria have been reported to self-assemble on the surface of substrates such as silicones, metals, high molecules, etc. (Moll et al., PNAS, 99:14646, 2002; Shenton et al., Nature, 389:585, 1997; Kuen et al., J. Bacterial., 179:1664, 1997).

Recently, the inventors of the present invention have prepared biosensors by wrapping CNT with carbohydrates using enzyme reaction, and binding bio-receptors to the water-soluble CNT wrapped with carbohydrates (PCT/KR03/02164). In such biosensor, the binding ability of carbohydrates to the surface of CNT is enhanced and the orientation of carbohydrates is well controlled but the wrapping efficiency of carbohydrates is undesirably low.

### SUMMARY OF THE INVENTION

Accordingly, the present inventors have exerted all possible efforts to develop a water-soluble CNT with high efficiency of wrapping, and confirmed that purified SLP can be stably wrapped around a CNT with high orientation by self-assembling, thereby completing the present invention.

The present invention relates to a water-soluble carbon nanotube wrapped with self-assembly material, and a method of preparation thereof.

The present invention also relates to a biosensor in which various kinds of bioreceptors are attached to the water-soluble CNT wrapped with self-assembly materials, and a method of preparation thereof.

The present invention also relates to a method for detecting various target bio-substances binding to or reacting with various receptors, using the above biosensors.

Other aspects, features and advantages of the invention will be more fully apparent from the ensuing disclosure and appended claims.

### BRIEF DESCRIPTION OF THE DRAWINGS

FIG. 1 schematically shows a self-assembly process of supramolecules. FIG. 1a shows that disc-shaped dendrimers (1) and fan-shaped supramolecules (2) are self-assembled into cylindrical structures (3) which are then arranged into three-dimensional hexagonal structures (4). FIG. 1b shows that cone-shaped molecules (5) are self-assembled into spherical structures (6), which are arranged into three-dimensional regular structures (7).

FIG. 2 shows a SDS-PAGE result of purified SLP.

FIG. 3 is a schematic diagram showing a method for wrapping CNTs with SLP.

FIG. 4 and FIG. 5 are images showing the result of atomic force microscopy (AFM) analysis for the CNTs wrapped with SLP.

### DETAILED DESCRIPTION OF THE INVENTION, AND PREFERRED EMBODIMENT THEREOF

The present invention provides a method for preparing a water-soluble CNT wrapped with self-assembly material which comprises the steps of: (a) providing a mixture of the self-assembly material and CNT; and (b) treating the mixture in a condition for inducing a self-assembly of the self-assembly material on CNT, thereby wrapping the CNT with the self-assembly material.

In the present invention, the self-assembly material is preferably SLP or SLP subunits, the SLP is preferably derived from a bacterium such as Aeromonas salmonicida, Aeromonas hydrophila, Bacillus stearthermophilus, Acetogenium kivui, Azotobacter vinelandii, Bacillus brevis, Bacillus polymyxa, Bacillus sphaericus, Caulobacter crescentus, Clostridium aceticum, Clostridium thermohydrosulfuricum, Clostridium thermosaccharolyticum, Comamonas acidovorans, Delftia acidovorans, Deinococcus radiodurans, Geobacillus stearothermophilus, Phormidium uncinatum, Sporosarcina ureae, Thermoanaerobacter kivui, Thermoanaerobacter thermoydrosulfuricus, or Thermoanaerobacterium thermosaccharolyticum, or an Archae microorganism such as Acidianus (Sulfolobus) brierleyi, Archaeoglobus fulgidus, Desulfurococcus mobilis, Desulfurolobus ambivalens, Halobacterium halobium (salinarum), Halobacterium volcanii, Hyperthermus botylicus, Methanoplanus limicola, Pyrobaculum islandicum, Pyrobaculum organotrophum, Pyrodictium brockii, Pyrodictium occultum, Sulfolobus acidocaldarius, Sulfolobus shibatae, Sulfolobus solfataricus, Staphylothermus marinus, Thermococcus celer, or Thermoproteus tenax. More preferably, the SLP is derived from Geobacillus stearothermophilus.

The present invention also provides a water-soluble CNT wrapped with self-assembly material, and produced by the above method.

The present invention also provides a method for fabricating a biosensor, wherein a receptor reacting with or binding to a target bio-substance or organic compound is attached to the water-soluble CNT wrapped with the self-assembly material.

The present invention also provides a biosensor, wherein a receptor reacting with or binding to a target bio-substance or organic compound is attached to the CNT wrapped with self-assembly material, and fabricated by the above method. The present invention also provides a method for detecting a target bio-substance or organic compound binding to or reacting with the receptor, which is characterized by using the above biosensor.

The present invention also provides a water-soluble CNT wrapped with SLP or SLP subunit material, and produced by the above method. The present invention also provides a method for fabricating a biosensor, wherein a receptor reacting with or binding to a target bio-substance or organic compound is attached to the water-soluble CNTs wrapped with SLP or SLP subunit material.

The present invention also provides a biosensor, wherein a receptor reacting with or binding to a target bio-substance or organic compound is attached to the CNT wrapped with SLP or SLP subunit material, and fabricated by the above method. The present invention also provides a method for detecting a target bio-substance or organic compound binding to or reacting with a receptor, which is characterized by using the above biosensor.

The target bio-substance or organic compound which is used in the present invention is a substance capable of acting as a target and being detected by reacting with or binding to the receptor, and preferably, a protein, a nucleic acid, an antibody, an enzyme, a carbohydrate, a lipid or any of various other biomolecules, and more preferably, a protein related to diseases.

The receptor which is used in the present invention is preferably an enzyme substrate, a ligand, an amino acid, a peptide, a protein, a nucleic acid, a lipid, a cofactor or a carbohydrate.

As used herein, the term “self-assembly material” refers to a material that is induced by non-covalent bond (hydrogen bond, ion bond, van der Waals bond, hydrophobic bond, electrostatic bond, etc.) to be assembled on a support material, which includes lipid, protein, peptide, DNA, RNA, and other organic materials. The materials formed by self-assembling have regular orientation and structure, and maintain functions. Examples of well-known self-assembly materials include lipid bi-layer, liposome, virus coat protein, SLP, DNA, RNA, etc., and also include supramolecules which are self-assembled on CNTs and exhibit water-solubility.

Examples of the self-assembling supramolecules include disc-shaped dendrimers (1), fan-shaped supramolecules (2), stick-chain shaped or cone-shaped molecules (5). An example of the fan-shaped supramolecules includes a compound of the following formula 1, an example of the disk-shaped supramolecules includes a compound of the following formula 2, and an example of the cone-shaped supramolecules includes a compound of the following formula 3:

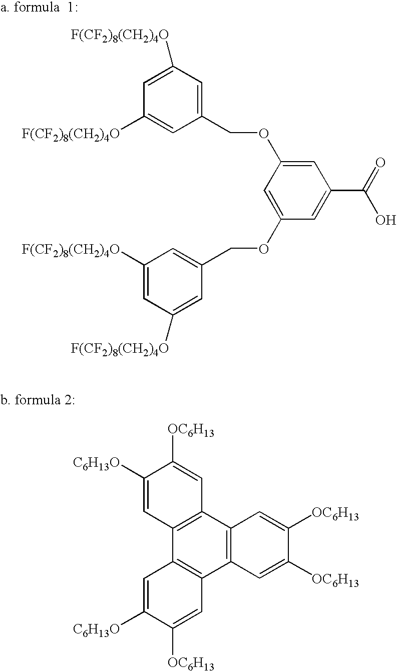

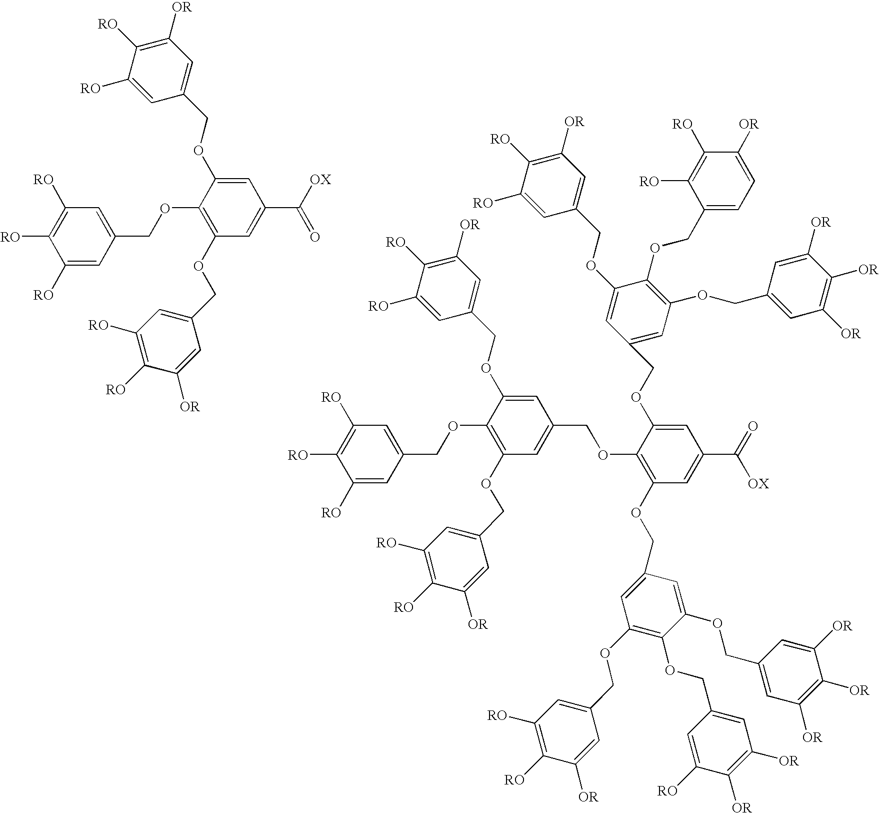

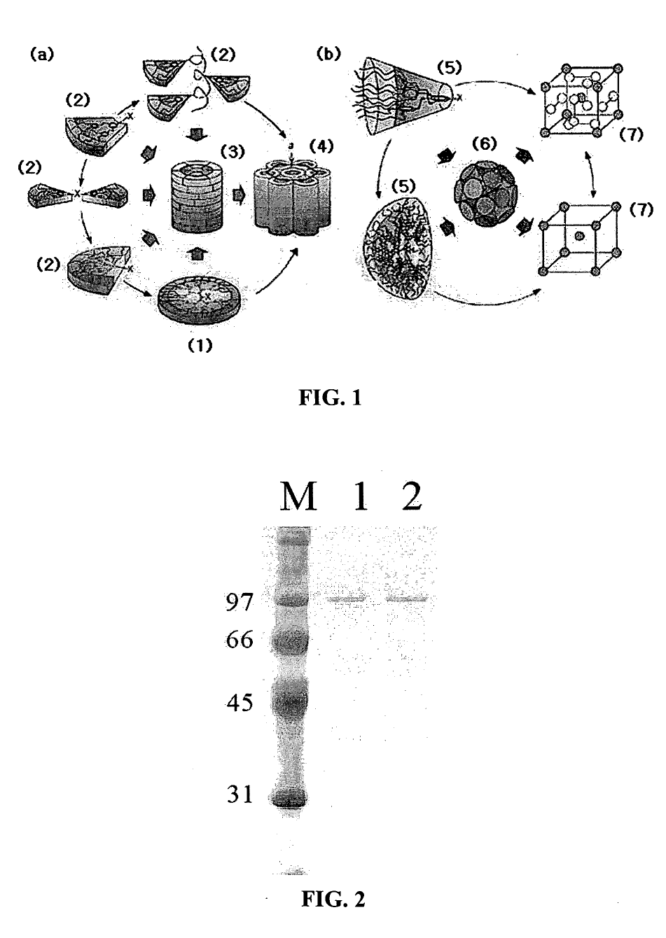

Such supramolecules are formed into a regular structure by physical secondary binding, such as van der Waals forces, unlike polymers where monomers are covalently bonded. Such supramolecules are self-assembled by suitable temperature or concentration, external magnetic field or electric field, etc, to form certain fine structures. As shown in FIG. 1a, such fan-shaped dendrimers are self-assembled into plate-shaped structures (1), which are then assembled into pillar-shaped structures (3), which are formed into a three-dimensional hexagonal structure (4). In addition, as shown in FIG. 1b, the cone-shaped supramolecules (5) are self-assembled into spheres (6), which are then arranged into a three-dimensional regular structure (7).

In the present invention, the term “wrapping” is defined to include the concept of the self-assembly material being self-assembled on CNT to encompass the surface of CNT by non-covalent bond.

As used herein, the term “biosensor” is intended to encompass structures and arrangements in which the receptors reacting with or binding to the bio-substances are attached to the CNT wrapped with self-assembling materials. The term “biosensor” is defined to include biochips.

As used herein, the term “bio-substance” is defined as the general name for substances derived from a living body, including nucleic acids, proteins, peptides, amino acids, enzyme substrates, ligands, cofactors, carbohydrates, lipids, oligonucleotides, RNA, and the like.

Hereinafter, the present invention will be described in more detail.

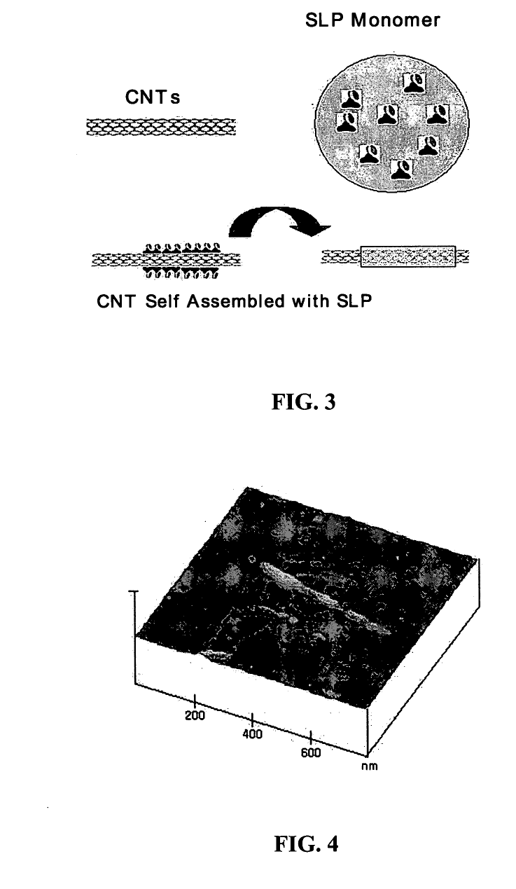

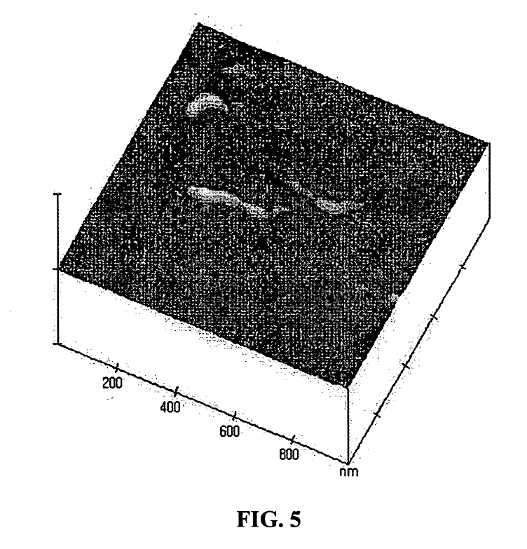

According to a preferred embodiment of the present invention, purified SLP was added to the CNT suspended in citrate buffer (pH 4.0) and self-assembly reaction was performed to prepare CNT wrapped with SLP (FIG. 3). The presence of the reaction product was observed by AFM image (FIG. 4, FIG. 5).

SLP purified from microorganisms has a characteristic of self-assembling on various hydrophobic substrates easily. Water-soluble CNT wrapped with SLP can be prepared using such characteristic. The self-assembling wrapping layer on the CNT may include SLP and/or SLP subunits of any suitable type.

The conventional method can be used for attaching receptors which react with or bind to target biomaterials, to the formed CNT wrapped with SLP, which are, for example, methods such as the cross-linking of self-assembled SLP on a substrate surface using glutaldehyde (Breitwieser et al., BioTechniques, 21:918, 1996; Gyorvary et al., Nano Lett., 3:315, 2003), and the fusion of streptavidin with the N terminal of SLP to express and induce the self-assembly reaction, thereby examining the biotin-avidin bond (Moll et al., PNAS, 99:14646, 2002).

Detecting the target bio-substances binding to or reacting with the receptor using the inventive biosensor can be performed by various conventional methods. The results of the detection reaction can be measured by a probe station that determines the electrical properties of the biosensors, and a fluorescent microscope that detects fluorescent dyes formed on the biosensors. Furthermore, a method is known previously in which radioactive isotopes are attached to reactants so that radiation can be measured by a radiation counter after reaction (Petrou et al., Biosens. Bioelectron., 17:859, 2002).

Methods allowing measurement in a liquid phase using such electrical properties include a method of employing oxidation-reduction reaction and electric charge accumulation. The oxidation-reduction reaction, which is a currently generalized electrochemical detection method, allows the measurement of a change in hydrogen or electrons by cyclic voltammetry, potentiometry, amperometry, and the like.

In a method of measuring the electric charge of ions charged in a bottom substrate in a liquid phase, the concentration of ions formed in CNT can be measured by forming an electrode on a top substrate forming a chip and measuring the charge of an electrode on a top substrate. In this case, the relationship between electrolyte and current is expressed as “the concentration of aqueous electrolyte solution ∝ the intensity of current”. Namely, since the distribution of electrolyte concentration according to the ion concentration of a reaction product formed on the CNT surface is in proportion to the intensity of current, the concentration of ions formed on the bottom substrate can be measured.

### EXAMPLES

The present invention will hereinafter be described in further detail by examples. It will however be obvious to a person skilled in the art that these examples can be modified into various different forms and the present invention is not limited to or by the examples. These examples are presented to further illustrate the present invention.

Especially, the present invention described SLP as a self-assembly material but any material which is self-assembled on CNT to show water solubility can be used without limitation. Moreover, it is possible to use a mixture containing such self-assembly materials and it is included in the scope of the present invention

Furthermore, while the examples herein are directed to water-soluble CNT wrapped with SLP, it will nonetheless be obvious to a person skilled in the art to implement the invention in a broad manner, e.g., to fix receptors such as enzyme substrate, ligand, amino acid, peptide, protein, RNA, PNA, lipid, cofactor, carbohydrate, etc. on the SLP of the water-soluble CNT wrapped with the such SLP to fabricate a biosensor, and then to detect easily target biomaterials or organic compounds which bind to or react with such receptors, using the fabricated biosensor.

### Example 1

### Preparation of SLP

Firstly, the production of SLP was confirmed in various bacteria (Aeromonas salmonicida, Aeromonas hydrophila, Geobacillus stearthermophilus, Bacillus stearthermophilus, etc.) to find new bacteria capable of effectively producing SLP which can be nano self-assembled. As a result of SDS-PAGE analysis of various cultured species, it was revealed that the most efficient species in respect of SLP production is Geobacillus stearothermophilus, which can be cultured at high temperature.

For mass production of SLP, Geobacillus stearthermophilus (KCTC 2107) was cultured at high concentration. The above species is one of hyperthermophilic gram positive bacteria whose appropriate culturing temperature is 55° C., which is very high. Such a hyperthermophilic bacterium has not been cultured at high density. The high density culture was performed using R/2 medium at 55° C. Feeding solution containing 500 g/L glucose, 50 g/L yeast extract, and 15 g/L MgSO₄.7H₂O was provided in DO-stat type, and the glucose concentration was maintained below 1 g/L to prevent catabolic repression caused by glucose and cell lysis. The cell density was around 10 g/L 65 hours after culturing, and such result is the highest density culture of hyperthermophilic bacteria among ones cultured to date. The level of SLP was analyzed by SDS-PAGE and the result showed the production efficiency of over 30% of total protein.

For isolating SLP, firstly cell was completely homogenized using french press (APV system, United Kingdom), and then pre-treated with 0.5% Triton X-100 for 1 hour. SLP was extracted at 4° C. for 2 hours by dissolving the pre-treated cell in 5M GuHCl and then the cell was centrifuged (40,000 g) to remove outer membrane. Finally the supernatant was dialyzed to isolate pure SLP very efficiently. 64 μl of the obtained SLP containing solution was mixed with 16 μl of SDS-PAGE sample buffer (60 mM Tris-HCl containing 25% glycerol, 2% SDS, 14.4 mM 2-mercaptoethanol and 0.1% bromophenol blue), and the mixture solution was boiled for 10 min. Then the solution was SDS-PAGE gel electrophoresed in 12% isolation gel. After electrophoresis, the gel was left in a dying solution (40% of methanol, 10% of acetic acid and 0.25 g/L of coomassie brilliant blue R) for 2 hours or more to be dyed. The gel was put in destaining solution (40% of methanol and 7% of acetic acid) for 2 hours or more twice to be destained.

In FIG. 2, lane M represents standard protein molecular weight, and lane 1 and 2 represent SLPs purified by the above method. As shown in FIG. 2, SLP was isolated and purified very efficiently.

### Example 2

### Preparation of CNT Wrapped with SLP

In the present invention, CNT from CarboLex Inc. was used after being isolated and purified (Rao et al., Science, 275:187, 1997). The purified SLP in the Example 1 was mixed with 100 μg of CNT which was pre-suspended in 20 mM citrate buffer (pH 4.0) to make the final concentration of the SLP 1 mg/mL, and then the mixture was left at room temperature for 10 hours to induce self-assembly reaction (FIG. 3). SLP has a property of self-assembling on hydrophobic CNT, so that water-soluble CNT wrapped with SLP can be fabricated easily without any special condition.

After completing reaction, the SLP-CNT reaction product was washed with distilled water for 5 min at 6000 rpm three times, and 20 μl of SLP-CNT reaction product was dropped on a silicone wafer. The wafer was centrifuged at 3000 rpm for 30 seconds under vacuum condition.

### Example 3

### Confirmation of CNT Wrapped with SLP

To confirm the presence of SLP-CNT reaction product prepared by biological self-assembly in the Example 2, the image was examined by the following method. AFM analysis was carried out with the NanoScope III MultiMode system (Digital Instruments) operated on tapping mode to be analyzed in the air, and a NanoProbe TESP was used as a tip. Typically, the contact mode is difficult to be applied in analysis of soft samples, so that the tapping mode is used to overcome the difficulty. Non-contact mode operation is also used, in which an image is obtained in consideration of the attraction between a sample and a probe. Occasionally the tapping mode is called a dynamic mode, and the non-contact mode includes both non-contact mode and dynamic mode. At the time of analyzing, frequency was 240-280 kHz and scanning rate was 1.97 Hz.

FIG. 4 and FIG. 5 are the images showing the result of AFM analysis of CNT wrapped with SLP. The scanning size was 0.8 μm and scanning rate was 0.7825 Hz. The images of FIG. 4 and FIG. 5 show that the SLP wrapping a CNT of about 650 nm length covers the CNT.

As described above in detail, the present invention provides water-soluble CNT wrapped with self-assembly material and a method of preparation thereof. The CNT wrapped with self-assembly material according to the present invention shows water solubility to have excellent applicability in comparison with usual CNT.

In various preferred embodiments of the invention, biosensors can be fabricated by attaching various receptors to the above-described water-soluble CNT wrapped with self-assembly material. Target biomaterials or organic compounds which bind to or react with such receptors can be easily detected using the biosensors.

While the present invention has been described with reference to particular illustrative embodiments, it is not to be restricted by such embodiments but only by the appended claims. It is to be appreciated that those skilled in the art can change or modify such embodiments without departing from the scope and spirit of the present invention.

What is claimed is:

1. A method for preparing a water-soluble CNT wrapped with a self-assembly material which comprises the steps of:
(a) providing a mixture of the self-assembly material and CNT; and
(b) treating the mixture in a condition for inducing a self-assembly of the self-assembly material on CNT, thereby wrapping the CNT with the self-assembly material.

2. The method according to claim 1, wherein the self-assembly material comprises SLP or SLP subunit.

3. The method according to claim 2, wherein the SLP comprises SLP derived from a bacterium selected from the group consisting of Aeromonas salmonicida, Aeromonas hydrophila, Bacillus stearthermophilus, Acetogenium kivui, Azotobacter vinelandii, Bacillus brevis, Bacillus polymyxa, Bacillus sphaericus, Caulobacter crescentus, Clostridium aceticum, Clostridium thermohydrosulfuricum, Clostridium thermosaccharolyticum, Comamonas acidovorans, Delftia acidovorans, Deinococcus radiodurans, Geobacillus stearothermophilus, Phormidium uncinatum, Sporosarcina ureae, Thermoanaerobacter kivui, Thermoanaerobacter thermoydrosulfuricus, and Thermoanaerobacterium thermosaccharolyticum.

4. The method according to claim 2, wherein the SLP comprises SLP derived from an Archae microorganism selected from the group consisting of Acidianus (Sulfolobus) brierleyi, Archaeoglobus fulgidus, Desulfurococcus mobilis, Desulfurolobus ambivalens, Halobacterium halobium (salinarum), Halobacterium volcanii, Hyperthermus botylicus, Methanoplanus limicola, Pyrobaculum islandicum, Pyrobaculum organotrophum, Pyrodictium brockii, Pyrodictium occultum, Sulfolobus acidocaldarius, Sulfolobus shibatae, Sulfolobus solfataricus, Staphylothermus marinus, Thermococcus celer, and Thermoproteus tenax.

5. The method according to claim 2, wherein the SLP comprises SLP derived from Geobacillus stearothermophilus.

6. A water-soluble CNT wrapped with a self-assembly material, and produced by the method of claim 1.

7. A water-soluble CNT wrapped with SLP or SLP subunit, and produced by the method of claim 2.

8. A method for fabricating a biosensor, wherein a receptor reacting with or binding to a target bio-substance or organic compound is attached to the water-soluble CNT wrapped with the self-assembly material according to claim 6.

9. The method according to claim 8, wherein the receptor comprises a receptor selected from the group consisting of enzyme substrates, ligands, amino acids, peptides, proteins, nucleic acids, lipids, cofactors and carbohydrates.

10. A method for fabricating a biosensor, wherein a receptor reacting with or binding to a target bio-substance or organic compound is attached to the water-soluble CNT wrapped with SLP or SLP subunit according to claim 7.

11. The method according to claim 10, wherein, the receptor comprises a receptor selected from the group consisting of enzyme substrates, ligands, amino acids, peptides, proteins, nucleic acids, lipids, cofactors and carbohydrates.

12. A biosensor, wherein a receptor reacting with or binding to a target bio-substance or organic compound is attached to the CNT wrapped with self-assembly material, and fabricated by the method of claim 8.

13. The biosensor according to claim 12, wherein the receptor comprises a receptor selected from the group consisting of enzyme substrates, ligands, amino acids, peptides, proteins, nucleic acids, lipids, cofactors and carbohydrates.

14. A biosensor, wherein a receptor reacting with or binding to a target bio-substance or organic compound is attached to the CNT wrapped with SLP or SLP subunit, and fabricated by the method of claim 10.

15. The biosensor according to claim 14, wherein, the receptor comprises a receptor selected from the group consisting of enzyme substrates, ligands, amino acids, peptides, proteins, nucleic acids, lipids, cofactors and carbohydrates.

16. A method for detecting a target bio-substance or organic compound binding to or reacting with a receptor, which is characterized by using the biosensor of claim 12.

17. The method according to claim 16, wherein the target bio-substance or organic compound comprises a target bio-substance or organic compound selected from the group consisting of proteins, nucleic acids, antibodies, enzymes, carbohydrates, lipids and other biomolecules.

18. A method for detecting a target bio-substance or organic compound binding to or reacting with a receptor, which is characterized by using the biosensor of claim 14.

19. The method according to claim 18, wherein the target bio-substance or organic compound comprises a target bio-substance or organic compound selected from the group consisting of proteins, nucleic acids, antibodies, enzymes, carbohydrates, lipids and other biomolecules.

20. A water-soluble CNT wrapped with a self-assembly material comprising SLP and/or subunits thereof.
